(19) United States
(12) Patent Application Publication
(10) Pub. No.: US 2025/0032297 A1
(43) Pub. Date: Jan. 30, 2025

(54) HYBRID ORTHOTIC SYSTEM

(71) Applicant: SubioMed, Inc., Bloomington, MN (US)

(72) Inventor: Barry A. Butler, Owatonna, MN (US)

(21) Appl. No.: 18/783,408

(22) Filed: Jul. 24, 2024

Related U.S. Application Data
(60) Provisional application No. 63/529,070, filed on Jul. 26, 2023.

(51) Int. Cl. A61F 5/01

(52) U.S. Cl. CPC A61F 5/0127; A61F 5/0111

(57) ABSTRACT

Systems and methods are disclosed including a tri-layer orthotic comprising an upper layer, a base layer, and a mid layer configured to suspend the upper layer over the base layer. A first receptacle can receive and couple the upper layer to the mid layer. A second receptacle can receive and couple the base layer to the mid layer. A hybrid orthotic system can include a support coupled to the upper layer of the tri-layer orthotic configured to secure a lower leg portion of a patient to the upper layer. An orthotic system can include a tuning element configured to couple to at least one of the upper layer, the base layer, or the mid layer. The orthotic system can include a tray for holding the tuning element prior to coupling the tuning element to the tri-layer orthotic.

### CLAIM OR PRIORITY

This application claims the benefit of priority under 35 U.S.C. § 119 (c) to U.S. Provisional Patent Application Ser. No. 63/529,070, filed on Jul. 26, 2023, which is incorporated by reference herein in its entirety.

### TECHNICAL FIELD

This document relates generally to orthotic systems that are configured to absorb and return energy during the gait cycle.

### BACKGROUND

The gait cycle is the cyclic pattern of movement that occurs during locomotion, such as walking or running. The gait cycle can be broken down into two main phases: stance and swing. The stance phase is the period where the foot is in contact with the ground and equates to 60% of the cycle when walking. The swing phases make up the remaining 40%. During the swing phase, the foot is free to move forward.

The stance phase can be further divided into five sub-phases: heel strike, foot flat, mid-stance, heel off, and toe off. The swing phase can also be divided into three sub-phases: acceleration, mid-swing, and deceleration.

Hard surfaces in modern human environments have changed the forces encountered by the human musculoskeletal system during the gait cycle as compared to the forces which it evolved to sustain. Impact energies from such surfaces enter the body through boney and dense tissues and through soft and fatty tissues, frequently causing physical damage leading to injury, in particular injury of the foot or ankle.

However, injury to the foot or ankle often impact other areas of the body, including the knee, hip, or lower back, and vice versa. Functional orthotics can improve patient motion. There is a need, to improve patient health and quality of life, for orthotic systems to correct deformities resulting from physical and other injuries to the foot, to address underlying pathologies and patho-mechanical foot dysfunctions, to accurately and precisely position the foot throughout the gait cycle, and to promote proper function and alignment and mitigate excessive forces.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1B illustrate different views of a first example tri-layer orthotic.

FIG. 2 illustrates a second example tri-layer orthotic having separate configurable components.

FIGS. 3A-3F illustrate different views of a third example tri-layer orthotic having separate configurable components.

FIGS. 4-8 example hybrid orthotic systems including a tri-layer orthotic having a support to attach to a leg of a patient.

FIGS. 9-11 illustrates an example hybrid orthotic having a removeable support.

FIGS. 12A-12C and 13A-13B illustrate example tri-layer orthotic systems having one or more configurable tuning elements.

FIGS. 14A-14C illustrate an example tray for holding configurable tuning elements.

### DETAILED DESCRIPTION

Ankle-foot orthosis (AFO) devices are specialized braces used to support and control the movement of the ankle and foot. AFOs come in different types and have different uses depending on the patient's needs. AFO devices are used for a variety of medical conditions such as cerebral palsy, stroke, multiple sclerosis, and spinal cord injuries.

One common type of AFO is the solid AFO. The solid AFO is a device made of rigid plastic that typically covers a bottom of a foot and at least a portion of a lower leg or calf of a patient to control, among other conditions, foot drop, toe walking, or one or more other conditions. Foot drop is a condition that causes the foot to drag while walking. The solid AFO holds the foot in a neutral position to prevent the foot from dropping. Toe walking is a gait abnormality where the patient walks on their toes. The solid AFO can help patients walk with a more natural gait pattern to reduce the risk of falls.

Other AFOs include the articulated AFO, the posterior leaf spring AFO, ground reaction AFO, etc. The articulated AFO has hinges at the ankle to allows the foot to move more freely, providing stability to improve patient balance while walking. The posterior leaf spring AFO has a support (e.g., a strip of plastic, etc.) that runs along the back of the leg and under the foot to support the ankle and foot during the swing phase of walking, preventing the foot from dropping, while still allowing for some movement of the ankle. The ground reaction AFO generally includes a wedge-shaped design that provides support to the foot and ankle to distribute the weight of the body more evenly to improve balance and reduce the risk of falls.

AFO devices benefit patients in several ways. They provide support and stability to the ankle and foot, which helps patients to maintain balance and walk more efficiently. AFOs also reduce the risk of falls, which is a significant concern for patients with mobility issues, improving patient quality of life by allowing them to perform daily activities.

The present inventors have recognized, among other things, that a dynamic orthotic suspension system to address foot pathologies that cause systemic pathologies (e.g., ankle, knee, and hip misalignment, etc.), such as disclosed in the commonly assigned Butler U.S. Pat. No. 9,066,559, titled “BI-LAYER ORTHOTIC AND TRI-LAYER ENERGY RETURN SYSTEM” and Butler U.S. patent application Ser. No. 18/084,322, titled “ENERGY RETURN ORTHOTIC SYSTEMS”, each of which incorporated herein by reference in their entireties, including the orthotic systems and tri-layer orthotics described therein, can be retrofit or otherwise incorporated with different AFO devices or braces to improve energy absorption, return, and movement through different portions of the gate cycle when using such devices or braces, further improving existing AFO devices and braces including those described above as well as, among others, ankle braces, walking boots, splints, foot braces, foot orthotics, etc.

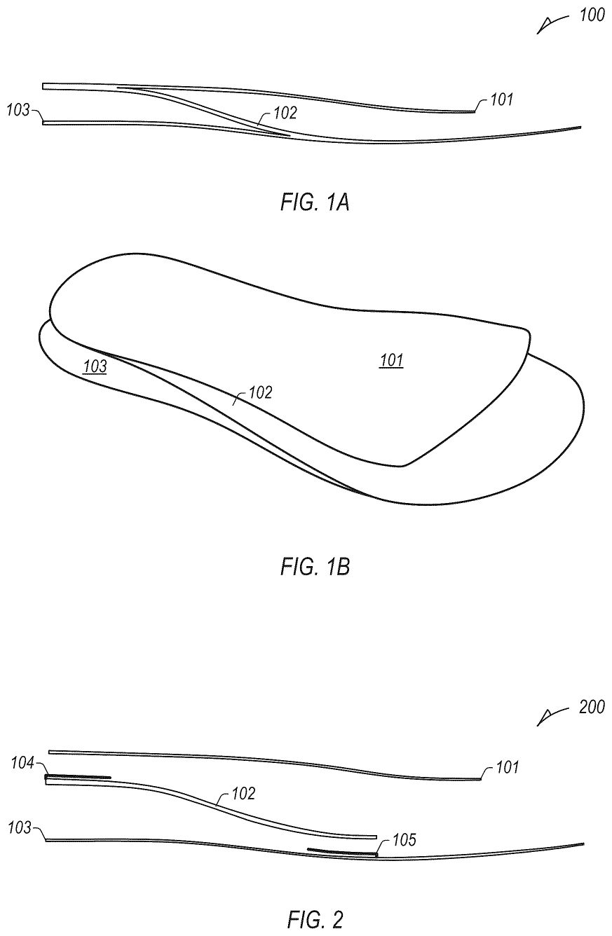

FIGS. 1A-1B illustrate different profile and perspective views of a first example tri-layer orthotic 100 having first, second, and third layers 101, 102, 103. The first layer 101 is an upper layer. The second layer 102 is a middle layer. The third layer 103 is a base layer. In an example, the third layer 103 extends the full length of the tri-layer orthotic 100 with the second layer 102 being the shortest layer between the first and third layers 101, 103. In another example, the third layer 103 extends from a proximal end under the heel portion of the tri-layer orthotic 100 to the second layer 102 and the second layer 102 extends from the first layer 101, such as from a heel portion of the first layer 101 to a distal toe portion of the second layer 102 extending the full length of the tri-layer orthotic 100.

The three layers form different spring or suspension areas, such as different rear, mid, and front spring sections. The rear spring section includes a heel portion on the first layer 101 suspended over a rear base portion of the third layer 103 to allow energy absorption and cushioning as well as ankle dorsiflexion at heel strike to offset plantar flexion at heel strike. Stored energy in the deflected material facilitates a smooth transition to mid-stance and the mid spring section of the second layer without foot slap and jarring decreasing the pronatory forces of ground impact. The elevation angle, dictated by the weight of the individual and the shape and stiffness of the different layers, optionally adjusted by a wedge, fulcrum, or one or more inserts, creates travel for smooth shock absorption, reducing jarring forces at heel strike.

During heel strike and mid-stance, the front spring section as well as dorsiflexion of the foot causes suspension of the ball of the foot on the first layer 101 over the second or third layers 102, 103. Forefoot loading of the gait cycle causes compression of the front spring section, causing the front of the first layer 101 to contact one or both of the second and third layers 102, 103, such that the foot is fully supported by the combination of the first and second layers 101, 102 or the first and third layers 101, 103.

In certain examples, the different layers can be formed from the same or different materials (e.g., carbon fiber, resin, or one or more other materials) having a relatively high or desired stiffness, tensile strength, strength to weight ratio, etc., as well as different or configurable shapes, profiles, sizes, or thicknesses. In an example, the tri-layer orthotic 100 or different layers of the tri-layer orthotic 100 can be vacuum formed from resin, baked, and trimmed to appropriate size, or formed from carbon fiber (e.g., wet lay-up with fiber laid into a mold and resin applied, prepreg lamination, resin transfer molding, etc.) or one or more other materials, etc. In other examples, certain layers or features can be designed having a desired amount of flexion at different levels of force. The different layers can be formed together or in different combinations or permutations, such as while forming the different layers, or in other examples coupled or joined (e.g., laminated, taped, adhered, physically joined, etc.) in various configurations or combinations after forming one or more of the different layers or combinations of layers.

By simulating the mobile adaptor function of the foot as it attacks the ground or uneven surfaces during the gait cycle the suspension of the foot decreases the necessary reactive forces and angular deflections the body has to absorb. By functionally adding additional joint axis in appropriate areas to simulate ankle, subtalar and mid-tarsal motions, better biomechanical control of the foot and ankle may be achievable. The suspension of the foot may facilitate smoother transition of energy such that the feel of ambulation is changed to that of a smooth rolling feel without jarring and shock. Decreased pronation, supination, ankle dorsiflexion and plantar flexion required for ambulation is expected. Resultant pathological forces may be mitigated. Restorative movement from use of the device in the case of individuals requiring bracing to limit motion due to pain/arthritis or people with fused or arthrodesed joints or prosthesis should facilitate more normal function and reduce the subsequent compensatory deterioration of adjacent structures. The line of progression should straighten during gait, resulting in better alignment during motion and decreased wear and tear on the body during gait. Less shock and jar of heel strike impact should positively influence the back and its pathologies. Control of pathological deflection of the tibia should decrease knee and hip joint wear and tear over time slowing arthritic changes.

For example, each of the three layers 101, 102, 103 can be formed individually then coupled or joined. In another example, two layers or a combination of portions of two layers can be formed, then coupled or joined with a separate portion of one or more layers.

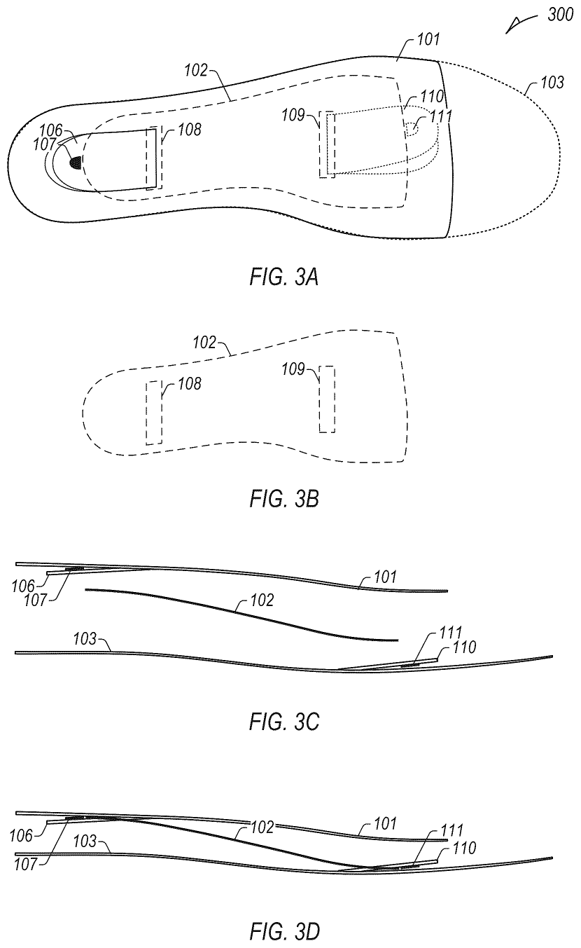

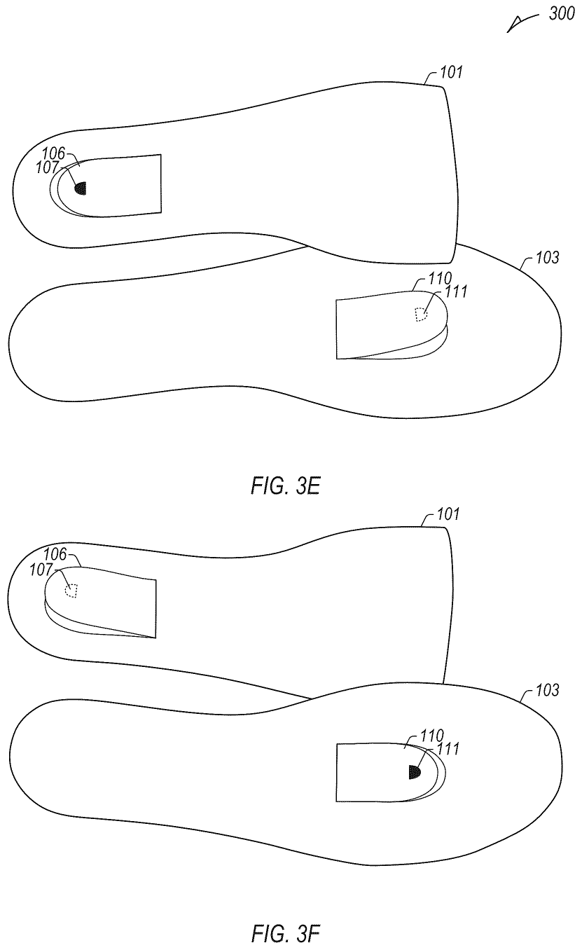

FIGS. 2 and 3A-3F illustrate improvements to the tri-layer orthotic 100 illustrated in FIGS. 1A-1B. For example, FIG. 2 illustrates a second example tri-layer orthotic 200 having separate configurable first, second, and third layers 101, 102, 103, similar to that described above, although at least partially mechanically joined by different mechanical features of the different layers, such as using a first receptacle 104 at a first end of the second layer 102 configured to receive a first end of the first layer 101 and a second receptacle 105 at a configurable position of the third layer 103 configured to receive a second end of the second layer 102. In other examples, the first layer 101 can include the first receptacle 104 to receive a first end of the second layer 102. In certain examples, the second layer 102 can include the second receptacle 105 configured receive a second end of the third layer 103.

In certain examples, the first and second receptacles 104, 105 can be open or closed at the sides (e.g., open on three sides or open on one side, etc.) or can include one or more mechanical features (e.g., stop features, teeth, ridges, etc.) to restrict or limit movement of the different layers or to maintain connection of the different layers once joined. In certain examples, although illustrated in FIG. 2 as grooves or pockets, one or more tracks, slots, slides, interlocking joints, snap fits, or other features at different positions on the different layers can combine to mechanically couple the different layers in different configurations or permutations, such as otherwise disclosed or shown herein. For example, different upper, middle, or base layers can be combined to form a single orthotic, with different layers having different profiles or properties (e.g., stiffness, flexion, etc.) selected for different outcomes or conditions (e.g., size, amount of prescribed or desired energy absorption or return, medical condition, gait, etc.).

In an example, in addition to the mechanic features illustrated in FIG. 2, one or more other mechanical features, such as rivets, snaps, interlocking joints, or one or more other mechanical retention features can be combined with the mechanical features to retain the different layers as a single orthotic. In other examples, one or more adhesives, tapes, or other laminate or epoxy can be added to the joined layers to form the single orthotic. Although described herein with respect to mechanical retention of the different layers, in other examples, such retention features or other retention features described herein can further correspond to other attachments, couplings, etc., such as of a support to the first layer 101, a tuning element, etc.

FIGS. 3A-3F illustrate different views of a third example tri-layer orthotic 300 having separate first, second, and third layers 101, 102, 103 having different receptacles, such as mechanical locking components (e.g., perforated locking tabs and corresponding slots, etc.). FIG. 3A illustrates a top view of the tri-layer orthotic 300. FIG. 3B illustrates a top view of the second layer 102 having first and second slots 108, 109. FIGS. 3C-3D illustrate exploded and assembled profile views of the tri-layer orthotic 300. FIGS. 3E-3F illustrate top and bottom views of the first and third layers 101, 103, respectively.

In this example, the second layer 102 includes first and second slots 108, 109 to receive and engage first and second tabs 106, 110 of the first and third layers 101, 103, respectively. The first layer 101 includes the first tab 106 to engage and retain the first slot 108 in the second layer 102 and the third layer 103 includes the second tab 110 to engage and retain the second slot 109 in the second layer 102. In certain examples, the first and second tabs 106, 110 can include perforated portions of different layers of the tri-layer orthotic 100. The first and second tabs 106, 110 can include respective lock portions 107, 111 to retain the first and second tabs 106, 110 in the first and second slots 108, 109 once assembled. In certain examples, the lock portions 107, 111 can include raised material, such as a lip or raised edge, providing an impediment or step for a slot to slide off a respective tab once joined.

As described above with respect to FIG. 2, mechanically coupling the different layers can provide a more configurable tri-layer orthotic having different profiles or properties selected for different outcomes or conditions, such as without having to manufacture or stock different static versions of each outcome in a final product. For example, different configurations of one or more of the first, second, or third layers can be selected to provide a single orthotic having one or more desired configurations or properties. One or more of the stiffness or shape of one or more of the first, second, or third layers can be selected or changed depending on a desired outcome or condition or based upon feedback by a user or observation by a caregiver. In addition, a user can try multiple variations of the product by replacing or combining different layers to form a single orthotic.

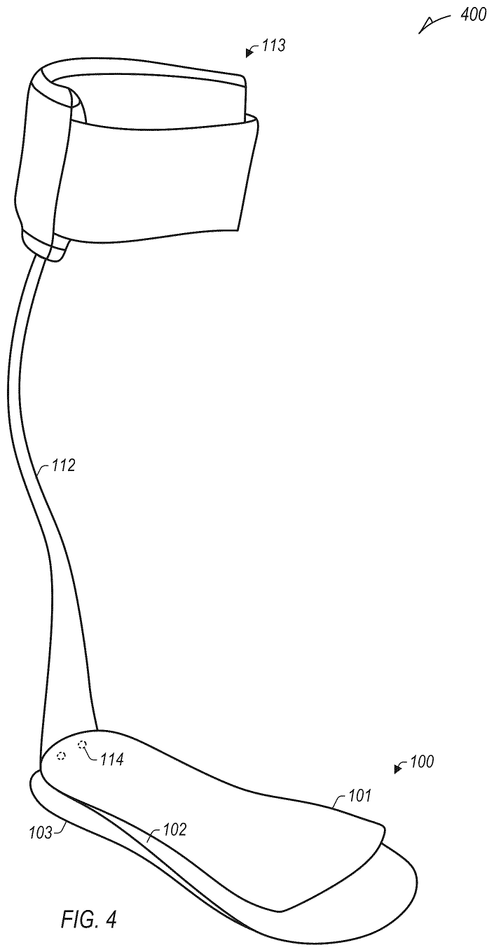

FIGS. 4-8 example hybrid orthotic systems including a tri-layer orthotic 100 having a coupled or attached support to attach to a foot or leg of a patient to assist or support the patient through at least a portion of the gait cycle. In contrast to taking existing AFO devices and adding the tri-layer orthotic 100 underneath, the present inventors have recognized that replacing the lower portion of existing AFO devices with the tri-layer orthotic 100 or adding support features to the upper layer of the tri-layer orthotic 100 can improve motion, support, and alignment through the gait cycle, providing the benefits described above while also providing therapeutic benefits of the AFO devices to the foot, ankle, lower leg, hips, back, or other anatomy of the patient, such as in contrast to only adding shock absorption under an existing AFO.

FIG. 4 illustrates an example hybrid orthotic system 400 including the tri-layer orthotic 100 and a support 112 (e.g., a rear support) attached to a first layer 101 of the tri-layer orthotic 100. In this example, the support 112 is configured to extend up and along a posterior side of a lower leg of the patient and attach to the lower leg using a fastener 113 including a posterior pad and strap. In other examples, the fastener 113 can include one or more other fasteners to attach the support 112 to or around the lower leg of the patient, such as including an anterior pad and strap or one or more other fasteners, for example, depending on a more different outcomes or conditions of the patient, etc.

The support 112 can include one or more other shapes or configurations, for example, to wrap around a lateral, medial, or anterior side of the lower leg, depending on a more different outcomes or conditions of the patient, etc. The support 112 can attach to the tri-layer orthotic 100 using one or more mechanical attachment features, such as one or more fasteners (e.g., rivets 114, etc.), other mechanical features (e.g., slots, grooves, tracks, slides, inserts, receptacles, etc.), or combinations thereof.

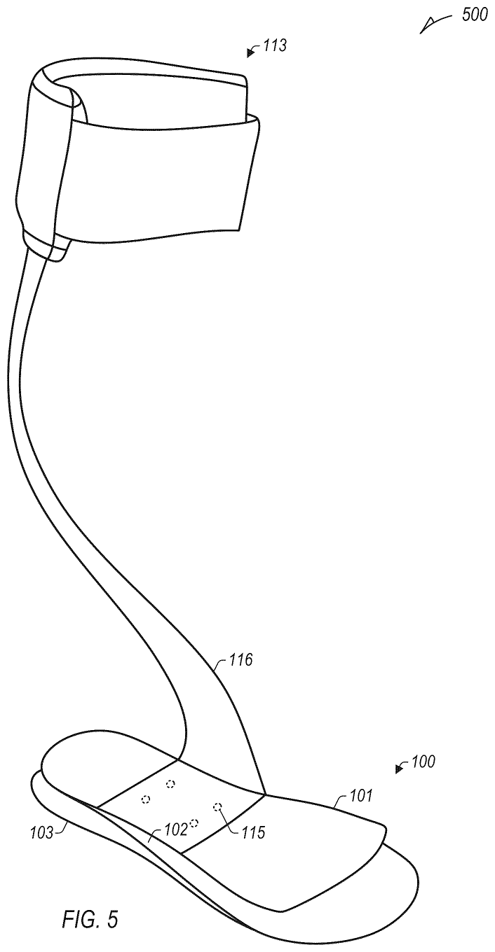

FIG. 5 illustrates an example hybrid orthotic system 500 including the tri-layer orthotic 100 and a support 116 (e.g., a medial support) attached to a first layer 101 of the tri-layer orthotic 100. In this example, the support 116 is configured to extend from the first layer 101 at the medial side of a foot of the patient up and around a posterior side of a lower leg of the patient and attach to the lower leg using a fastener 113.

The support 116 can include one or more other shapes or configurations, for example, depending on one or more different outcomes or conditions of the patient, etc. The support 116 can attach to the tri-layer orthotic 100 using one or more mechanical attachment features, such as one or more fasteners (e.g., rivets 115), (e.g., slots, grooves, tracks, slides, inserts, receptacles, etc.), or combinations thereof. For example, the support 116 can attach to the first layer 101 by coupling a first mechanical feature of one of the support 116 or the first layer 101 (e.g., a slide, an insert, etc.) with a corresponding second mechanical feature of the other of the support 116 or the first layer 101 (e.g., a receptacle, a groove, a slot, etc.).

Although illustrated as a medial support in the example of FIG. 5, the support 116 can be similarly attached to a lateral side of the of the tri-layer orthotic 100, or at one or more other portions of the upper layer of the tri-layer orthotic 100 using one or more different attachments, etc.

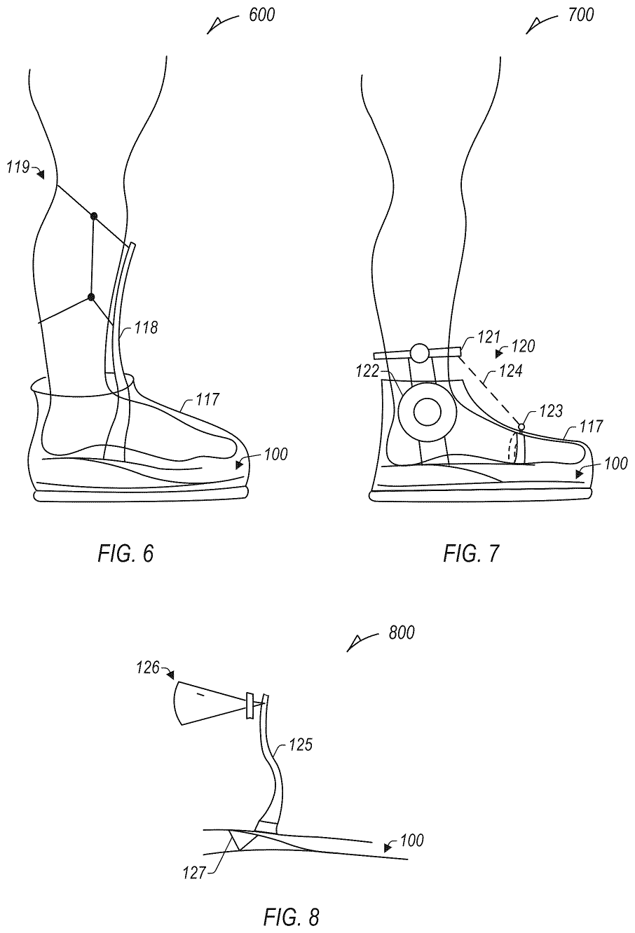

FIG. 6 illustrates an example profile view of a foot and lower leg of a patient and an example hybrid orthotic system 600 including the tri-layer orthotic 100 and a support 118 (e.g., an anterior support) attached to an upper layer of the tri-layer orthotic 100 in a shoe 117 of the patient. In this example, the support 118 is configured to extend along an anterior portion of a lower leg of the patient and attach to the lower leg using a fastener 119 (e.g., a calf strap, a posterior calf parachute, etc.). In other examples, one or more other fasteners 119, including pads or straps, can be used, depending on one or more different outcomes or conditions of the patient, etc.

FIG. 7 illustrates an example profile view of a foot and lower leg of a patient and an example hybrid orthotic system 700 including the tri-layer orthotic 100 and a hinged support 120 (e.g., a hinged drop foot brace) attached to an upper layer of the tri-layer orthotic 100 in a shoe 117 of the patient. The hinged support 120 includes a hinge 122 (e.g., an articulated joint, etc.) and a lower leg fastener 121, such as a pad and strap or corresponding fastener to attach to the lower leg of the patient at or above the ankle. The hinged support 120 additionally includes an optional drop-foot attachment 124, such as a cord, wire, rubber band, strap, or one or more other attachments between the lower leg fastener 121 and an anchor 123 coupled to the upper layer of the tri-layer orthotic 100, such as using a flange or other element, for example, to limit extension (or drop) of a foot of the patient, etc. In certain examples, the anchor 123 can perforate a lace hole or other opening of the shoe 117.

FIGS. 6 and 7 further illustrate that the tri-layer orthotic 100 has different heel and forefoot portions. The heel portion can include a rear portion of the first layer (or upper layer) configured to contact or support the heel of the patient. The forefoot portion can include the front portion of the first layer under the ball of the foot as well as the front portion of the third layer (or base layer) configured to contact at least a portion of one or more toes of the patient during at least a portion of the gait cycle. In certain examples, the second layer can include a distal toe portion configured to support at least a portion of one or more toes of the patient during at least a portion of the gait cycle. In an example, the base layer can optionally include only the portion of the third layer from the point of contact with the second layer and back, under the heel of the patient. In other examples, the second layer includes only that portion between the first and third layers, such as illustrated in FIGS. 1-2 and 3A-3F.

FIG. 8 illustrates an example profile view of an example hybrid orthotic system 800 including the tri-layer orthotic 100 and a support 125 attached to an upper layer of the tri-layer orthotic 100. The support 125 can include one or more shapes or lengths, for example, depending on a more different outcomes or conditions of the patient, etc., and can attach to a lower leg of a patient using a strap 126 or one or more other fasteners.

In certain examples, one or more other elements can be coupled to the tri-layer orthotic 100 to limit or modify the suspension of the tri-layer orthotic 100, such as a wedge 127 or one or more other elements (e.g., fulcrum, insert, etc.). In certain examples, such elements can create drop foot functionality, creating dorsiflexion of the forefoot in combination with the support 125 and strap 126, pulling the foot upright retrograde to the lower leg.

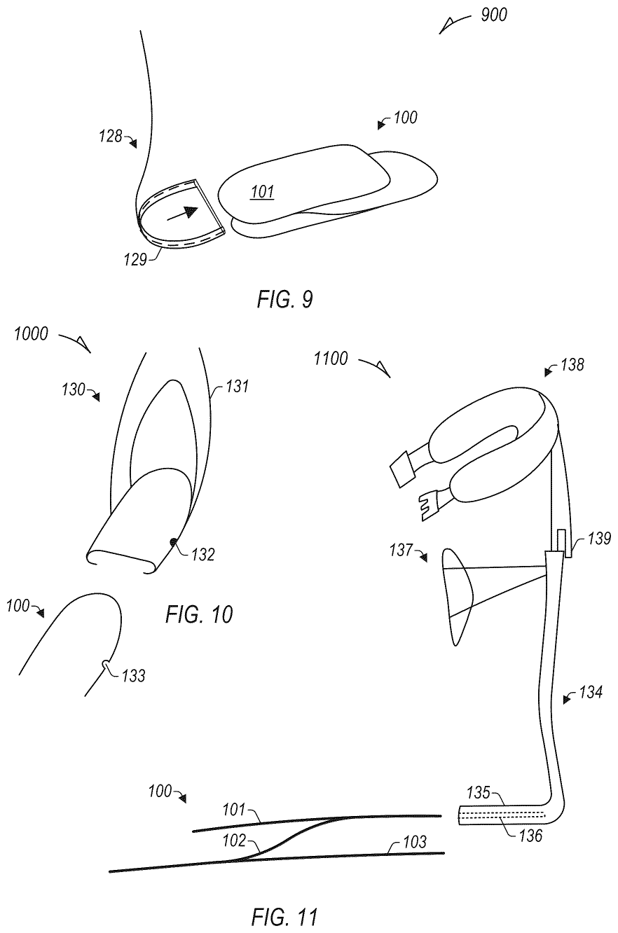

FIGS. 9-11 illustrate example hybrid orthotic system support attachments, including a tri-layer orthotic 100 or portion thereof and removeable support elements to mechanically couple to or separate from the tri-layer orthotic 100.

FIG. 9 illustrates an example hybrid orthotic system 900 including the tri-layer orthotic 100 and a support 128 including a groove 129 configured to couple to a first layer 101 of the tri-layer orthotic 100. In certain examples, the groove 129 can include one or more of a top and bottom surface to slide over and cover a top or bottom surface of the first layer 101 of the tri-layer orthotic 100.

In certain example, one or more additional elements can be coupled to the bottom surface, such as to limit or modify suspension of the tri-layer orthotic 100. In other examples, the top surface can include one or more elements to direct movement of the patient through a portion of the gait cycle or to secure a portion of a foot of the patient to the tri-layer orthotic 100, such as a heel cup above and around the groove 129, ankle or Achilles tendon support with an open heel, etc.

FIG. 10 illustrates an example hybrid orthotic system 1000 including the tri-layer orthotic 100 and a support 130 including a suspension element 131 (e.g., a heel cup, an Achilles tendon support with an open heel, etc.) to support a portion of an anatomy of the patient and a mechanical stop feature 132 to interact with a corresponding mechanical stop feature 133 in the tri-layer orthotic 100. The mechanical stop features 132, 133 can be configured to maintain the support 130 to the tri-layer orthotic 100 once mechanically coupled or engaged, such as by slidably attaching the support 130 to the tri-layer orthotic 100, etc.

FIG. 11 illustrates an example hybrid orthotic system 1100 including the tri-layer orthotic 100 having first, second, and third layers 101, 102, 103 and a support 134 including a groove 136 in a lower portion 135 of the support 134 configured to couple the support 134 to the first layer 101 of the tri-layer orthotic 100. The support 134 optionally includes or is attached to one or more fasteners, such as first and second fasteners 137, 138 configured to couple the support to a leg of a patient. In an example, the second fastener 138 include a pad, strap, and buckle configured to attach to the support 134 using one or more mechanical features, such as a connector 139 (e.g., a push-pin connector. In certain examples, the support 134 can include a heel cup coupled to the lower portion 135.

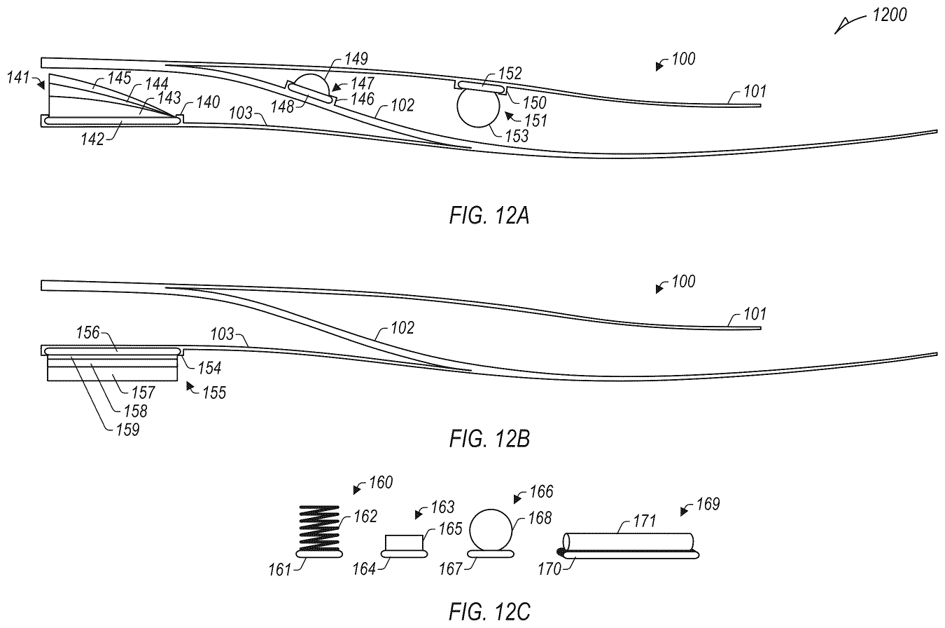

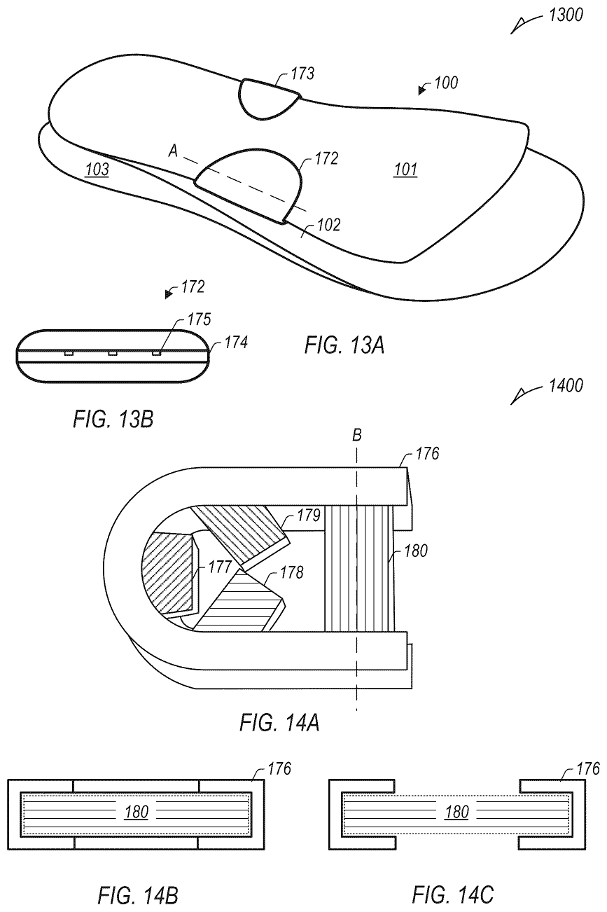

FIGS. 12A-12C and 13A-13B illustrate example tri-layer orthotic systems including a tri-layer orthotic 100 having one or more configurable tuning elements incorporated or positioned on or between different portions of one or more of the first, second, or third layers 101, 102, 103 of the tri-layer orthotic 100, such as coupled to one or more receptacles on the tri-layer orthotic 100.

FIGS. 12A-12B illustrate profile views of different tri-layer orthotic systems 1200 having different configurable tuning elements. FIG. 12A illustrates example first, second, and third tuning elements 141, 147, 151. The first tuning element 141 includes a first heel tuning element configured to reduce movement of a rear suspension of the tri-layer orthotic between. The first tuning element 141 can include one or more of optional wedges 143, 144, 145 having different thicknesses coupled to an insert 142 configured to slide into a receptacle 140 on the third layer 103 of the tri-layer orthotic 100. In other examples, the groove 140 can be located on the underside of the first or second layers 101, 102 instead of on top of the third layer 103.

The second and third tuning elements 147, 151 include different shape elements 149, 153 coupled to respective inserts 148, 152 configured to slide into respective receptacles 146, 150 at different locations on the first and second layers 101, 102. In other examples, one or more other shaped elements can be coupled to the different inserts, or the tuning elements can be positioned at one or more other locations. Although illustrated as raised with respect to the surface (e.g., top or bottom surface) of the first, second, and third layers 101, 102, 103 in FIG. 12A, in other examples, the receptacles can be flush with the surface, or one or more channels (e.g., an inverted T channel, etc.) or grooves can be formed in the surface of the tri-layer orthotic 100 for the inserts to engage with minimal impact to the surfaces of the tri-layer orthotic when the elements are not in use.

FIG. 12B illustrates an example fourth tuning element 155 including a heel tuning element configured to raise the heel height of the tri-layer orthotic 100. The fourth tuning element 155 can include one or more of optional elements 157, 158, 159 having different thicknesses coupled to an insert 156 configured to slide into a receptacle 154 under the third layer 103 of the tri-layer orthotic 100. In other examples, one or more different sized or shaped elements can couple to the groove 154, or the fourth tuning element 155 can be located at one or more other locations of the tri-layer orthotic 100 or can be used in combination with one or more other example tuning elements in various combinations or configurations.

FIG. 12C illustrates example fifth through eighth tuning elements 160, 163, 166, 169 having different optional configurations or shapes, composed of different materials having different shapes or properties, for example, depending on a more different outcomes or conditions of the patient, etc.

The fifth tuning element 160 includes a spring 162 coupled to an insert 161 configured to engage with one or more receptacles at one or more locations of the tri-layer orthotic 100, such as between two of the different layers, etc. The sixth tuning element 163 includes a block element 165 formed from one or more compliant or rigid materials, for example, depending on a more different outcomes or conditions of the patient, etc. The block element 165 can be coupled to an insert 164 configured to engage with one or more receptacles at one or more locations of the tri-layer orthotic 100.

The seventh tuning element 166 includes a circular element 168 (e.g., spherical, cylindrical, etc.) formed from one or more compliant or rigid materials, for example, depending on a more different outcomes or conditions of the patient, etc. The circular element 168 can be coupled to an insert 167 configured to engage with one or more receptacles at one or more locations of the tri-layer orthotic 100. The eighth tuning element 169 includes a cylindrical element 171 formed from one or more compliant or rigid materials, for example, depending on a more different outcomes or conditions of the patient, etc. The cylindrical element 171 can be coupled to an insert 170 configured to engage with one or more receptacles at one or more locations of the tri-layer orthotic 100.

In other examples, one or more other shapes or elements can be coupled to an insert, such as illustrated herein, and be coupled to one or more surfaces or edges or mechanical features of the tri-layer orthotic 100, such as to adjust, limit, or otherwise alter energy transfer or motion of a patient along at least a portion of a gait cycle.

FIG. 13A illustrates a perspective view of an example tri-layer orthotic system 1300 including a tri-layer orthotic 100 having first, second, and third layers 101, 102, 103 and ninth and tenth tuning elements 172, 173 (e.g., clam shaped tuning elements) configured to engage and couple to one or more of the different layers of the tri-layer orthotic 100. In certain examples, the different tuning elements can include lateral or medial lifts, such as a cuboid lift, etc.

FIG. 13B illustrates a cross section view of the ninth tuning element 172 along section A illustrated in FIG. 13A. The ninth tuning element 172 includes a major groove 174 configured to allow the tuning element to slide onto one or more layers of the tri-layer orthotic. In certain examples, the groove 174 can include one or more teeth or ridges 175 to engage corresponding slots or grooves in the tri-layer orthotic 100, such as to reduce movement of the ninth tuning element 172 with respect to the tri-layer orthotic 100 once engaged. Additionally, one or more mechanical stop features, such as that illustrated in FIG. 10, can be incorporated to further maintain the ninth tuning element 172 to the tri-layer orthotic 100 once mechanically coupled or engaged.

FIGS. 14A-14C illustrate an example tray system 1400 for holding one or more configurable tuning elements in an example tray 176. The tuning elements can include shims, inserts, wedges, or one or more other functional tuning elements, in certain examples, numbered, colored, or labeled for different pathologies or treatments, with corresponding labels or numbers on a corresponding tri-layer orthotic or orthotic system.

In certain examples, the tray 176 can include a groove or receptacle similar to or engageable with one or more corresponding features of the tri-layer orthotic illustrated herein. Although illustrated without a top or bottom, in certain examples, one or both can be included, closing the opening between the opposite sides of the tray 176 on at least one side. In certain examples, the tray 176 can include different tray elements, such as first-fourth tray elements 177, 178, 179, 180 having different shapes or profiles, each configured to engage a similar groove as the tray 176 on one or more portions of a corresponding tri-layer orthotic. For example, one or more of the tray elements can include one or more of a groove, slide, insert, or one or more other receptacles to engage a corresponding feature of the tri-layer orthotic. In other examples, the tray elements can be sized to engage with corresponding mechanical features in the tri-layer orthotic. For example, a first layer of the tri-layer orthotic can include a track or groove having a similar or the same profile as the tray 176.

One or more of the first-fourth tray elements 177, 178, 179, 180 can be inserted at various locations on the track or groove of the tri-layer orthotic. In certain examples, the different tray elements can include one or more identifiers, such as a number, label, color, etc. In other examples, the tray elements can include an adhesive, such that the different elements can be adhered to different locations (e.g., labeled, identified, etc.) of the tri-layer orthotic. In other examples, combinations of mechanical features, adhesives, or one or more other attachments mechanisms can be used to place and maintain one or more of the first-fourth tray elements 177, 178, 179, 180 to the tri-layer orthotic.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

Example 1 is a tri-layer orthotic system, comprising: an upper layer having a heel portion and a front portion; a base layer having a proximal end and a distal end; and a mid layer configured to connect to the base layer and the upper layer, wherein at least one of the upper layer and the mid layer includes a first receptacle to receive and couple the upper layer to the mid layer, wherein at least one of the base layer and the mid layer includes a second receptacle to receive and couple the base layer to the mid layer, and wherein the mid layer is configured to suspend the upper layer over the base layer when the mid layer is connected to the base layer and the upper layer.

In Example 2, the subject matter of Example 1, comprising: a forefoot portion opposite the heel portion, the forefoot portion comprising the front portion of the upper layer and at least a portion of one of the mid layer or the base layer, wherein the heel portion and the forefoot portion of the tri-layer orthotic system are configured to contact and support respective heel and forefoot portions of a foot of a patient through at least a portion of a gait cycle.

In Example 3, the subject matter of any of Examples 1-2, wherein the first receptacle includes a groove or pocket at a proximal end of the mid layer to receive the heel portion of the upper layer, and wherein the second receptacle includes a groove or pocket at or between one of a mid portion of the base layer or the distal end of the base layer to receive a distal end of the mid layer.

In Example 4, the subject matter of any of Examples 1-3, wherein the second receptacle includes a groove or pocket at the distal end of the mid layer to receive a distal end of the base layer.

In Example 5, the subject matter of any of Examples 1-4, wherein a distal toe portion of the mid layer extends distally beyond the front portion of the upper layer.

In Example 6, the subject matter of any of Examples 1-5, wherein the distal end of the base layer extends distally beyond the front portion of the upper layer.

In Example 7, the subject matter of any of Examples 1-6, wherein the tri-layer orthotic system is configured to control foot, ankle, and body biomechanics during the gait cycle.

In Example 8, the subject matter of any of Examples 1-7, wherein the mid layer includes a first slot between a mid portion of the mid layer and a proximal end of the mid layer and a second slot between the mid portion of the mid layer and a distal end of the mid layer, wherein the first receptacle includes the first slot and the second receptacle includes the second slot, wherein the upper layer includes a first tab between a mid portion of the upper layer and the heel portion of the upper layer, the first tab having an open end towards the heel portion of the upper layer, wherein the first slot is configured to engage the first tab to connect the mid layer to the upper layer, wherein the base layer includes a second tab between a mid portion of the base layer and the distal end of the base layer, the second tab having an open end towards the distal end of the base layer, wherein the second slot is configured to engage the second tab to connect the mid layer to the base layer, wherein the mid layer is configured to suspend the upper layer over the base layer when the first and second slots are engaged with the first and second tabs.

In Example 9, the subject matter of Example 8, wherein the mid layer has a length smaller than the base layer and the upper layer.

In Example 10, the subject matter of any of Examples 8-9, wherein the first tab is configured to deflect from the upper layer towards the base layer to receive the first slot of the mid layer and the second tab is configured to deflect from the base layer towards the upper layer to receive the second slot of the mid layer, wherein an upper surface of the first tab includes a first raised edge to retain the first slot of the mid layer over the first tab once the first slot is engaged with the first tab and a lower surface of the second tab includes a second raised edge to retain the second slot of the mid layer over the second tab once the second slot is engaged with the second tab.

Example 11 is a hybrid orthotic system, comprising: a tri-layer orthotic, comprising: an upper layer having a heel portion and a front portion; a base layer having a proximal end and a distal end; and a mid layer configured to connect to the base layer and the upper layer and to suspend the upper layer over the base layer when connected to the base layer and the upper layer; and a support having a first end coupled to the upper layer of the tri-layer orthotic, a mid portion extending away from an upper surface of the upper layer, and a second end configured to couple to and secure a lower leg portion of a patient to the upper layer of the tri-layer orthotic.

In Example 12, the subject matter of Example 11, wherein one of a distal toe portion of the mid layer extends distally beyond the front portion of the upper layer or the distal end of the base layer extends distally beyond the front portion of the upper layer.

In Example 13, the subject matter of any of Examples 11-12, wherein the support comprises an upper portion of an ankle-foot orthosis (AFO) coupled to the upper layer of the tri-layer orthotic system.

In Example 14, the subject matter of any of Examples 11-13, wherein the first end of the support is configured to couple to the heel portion of the upper layer.

In Example 15, the subject matter of any of Examples 11-14, wherein the first end of the support is configured to couple to a medial or lateral side of a mid portion of the upper layer.

In Example 16, the subject matter of any of Examples 11-15, wherein the second end includes a fastener to attach to the lower leg of the patient, including at least one of an ankle, tibia, fibula, or calf of the patient.

Example 17 is an orthotic system, comprising: a tri-layer orthotic, comprising: an upper layer having a heel portion and a front portion; a base layer having a proximal end and a distal end; and a mid layer configured to connect to the base layer and the upper layer and to suspend the upper layer over the base layer when connected to the base layer and the upper layer; and a tuning element configured to couple to at least one of the upper layer, the base layer, or the mid layer of the tri-layer orthotic.

In Example 18, the subject matter of Example 17, wherein the tuning element comprises a groove configured to engage at least one of the upper layer, the base layer, or the mid layer of the tri-layer orthotic.

In Example 19, the subject matter of any of Examples 17-18, wherein the tuning element comprises an insert configured to couple to a receptacle on at least one of the upper layer, the base layer, or the mid layer of the tri-layer orthotic.

In Example 20, the subject matter of Example 19, comprising: a tray for holding the tuning element prior to coupling the tuning element to at least one of the upper layer, the base layer, or the mid layer, wherein the tray includes a groove to hold a plurality of different configurable tuning elements, wherein an inner profile of the groove matches an inner profile of the receptacle on at least one of the upper layer, the base layer, or the mid layer of the tri-layer orthotic, wherein the tuning element is labeled with an identifier, and wherein the tri-layer orthotic comprises a corresponding label at the receptacle configured to receive the tuning element labeled with the identifier.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

The above detailed description is intended to be illustrative, and not restrictive. The scope of the disclosure should, therefore, be determined with references to the appended claims, along with the full scope of equivalents to which such claims are entitled.

What is claimed is:

1. A tri-layer orthotic system, comprising:
an upper layer having a heel portion and a front portion;
a base layer having a proximal end and a distal end; and
a mid layer configured to connect to the base layer and the upper layer,
wherein at least one of the upper layer and the mid layer includes a first receptacle to receive and couple the upper layer to the mid layer,
wherein at least one of the base layer and the mid layer includes a second receptacle to receive and couple the base layer to the mid layer, and
wherein the mid layer is configured to suspend the upper layer over the base layer when the mid layer is connected to the base layer and the upper layer.

2. The tri-layer orthotic system of claim 1, comprising:
a forefoot portion opposite the heel portion, the forefoot portion comprising the front portion of the upper layer and at least a portion of one of the mid layer or the base layer,
wherein the heel portion and the forefoot portion of the tri-layer orthotic system are configured to contact and support respective heel and forefoot portions of a foot of a patient through at least a portion of a gait cycle.

3. The tri-layer orthotic system of claim 1, wherein the first receptacle includes a groove or pocket at a proximal end of the mid layer to receive the heel portion of the upper layer, and
wherein the second receptacle includes a groove or pocket at or between one of a mid portion of the base layer or the distal end of the base layer to receive a distal end of the mid layer.

4. The tri-layer orthotic system of claim 1, wherein the second receptacle includes a groove or pocket at the distal end of the mid layer to receive a distal end of the base layer.

5. The tri-layer orthotic system of claim 1, wherein a distal toe portion of the mid layer extends distally beyond the front portion of the upper layer.

6. The tri-layer orthotic system of claim 1, wherein the distal end of the base layer extends distally beyond the front portion of the upper layer.

7. The tri-layer orthotic system of claim 1, wherein the tri-layer orthotic system is configured to control foot, ankle, and body biomechanics during a gait cycle.

8. The tri-layer orthotic system of claim 1,
wherein the mid layer includes a first slot between a mid portion of the mid layer and a proximal end of the mid layer and a second slot between the mid portion of the mid layer and a distal end of the mid layer,
wherein the first receptacle includes the first slot and the second receptacle includes the second slot,
wherein the upper layer includes a first tab between a mid portion of the upper layer and the heel portion of the upper layer, the first tab having an open end towards the heel portion of the upper layer, wherein the first slot is configured to engage the first tab to connect the mid layer to the upper layer,
wherein the base layer includes a second tab between a mid portion of the base layer and the distal end of the base layer, the second tab having an open end towards the distal end of the base layer, wherein the second slot is configured to engage the second tab to connect the mid layer to the base layer,
wherein the mid layer is configured to suspend the upper layer over the base layer when the first and second slots are engaged with the first and second tabs.

9. The tri-layer orthotic system of claim 8, wherein the mid layer has a length smaller than the base layer and the upper layer.

10. The tri-layer orthotic system of claim 8, wherein the first tab is configured to deflect from the upper layer towards the base layer to receive the first slot of the mid layer and the second tab is configured to deflect from the base layer towards the upper layer to receive the second slot of the mid layer,
wherein an upper surface of the first tab includes a first raised edge to retain the first slot of the mid layer over the first tab once the first slot is engaged with the first tab and a lower surface of the second tab includes a second raised edge to retain the second slot of the mid layer over the second tab once the second slot is engaged with the second tab.

11. A hybrid orthotic system, comprising:
a tri-layer orthotic, comprising:
an upper layer having a heel portion and a front portion;
a base layer having a proximal end and a distal end; and
a mid layer configured to connect to the base layer and the upper layer and to suspend the upper layer over the base layer when connected to the base layer and the upper layer; and
a support having a first end coupled to the upper layer of the tri-layer orthotic, a mid portion extending away from an upper surface of the upper layer, and a second end configured to couple to and secure a portion of a lower leg of a patient to the upper layer of the tri-layer orthotic.

12. The hybrid orthotic system of claim 11, wherein one of a distal toe portion of the mid layer extends distally beyond the front portion of the upper layer or the distal end of the base layer extends distally beyond the front portion of the upper layer.

13. The hybrid orthotic system of claim 11, wherein the support comprises an upper portion of an ankle-foot orthosis (AFO) coupled to the upper layer of the tri-layer orthotic system.

14. The hybrid orthotic system of claim 11, wherein the first end of the support is configured to couple to the heel portion of the upper layer.

15. The hybrid orthotic system of claim 11, wherein the first end of the support is configured to couple to a medial or lateral side of a mid portion of the upper layer.

16. The hybrid orthotic system of claim 11, wherein the second end includes a fastener to attach to the lower leg of the patient, including at least one of an ankle, tibia, fibula, or calf of the patient.

17. An orthotic system, comprising:
a tri-layer orthotic, comprising:
an upper layer having a heel portion and a front portion;
a base layer having a proximal end and a distal end; and
a mid layer configured to connect to the base layer and the upper layer and to suspend the upper layer over the base layer when connected to the base layer and the upper layer; and
a tuning element configured to couple to at least one of the upper layer, the base layer, or the mid layer of the tri-layer orthotic.

18. The orthotic system of claim 17, wherein the tuning element comprises a groove configured to engage at least one of the upper layer, the base layer, or the mid layer of the tri-layer orthotic.

19. The orthotic system of claim 17, wherein the tuning element comprises an insert configured to couple to a receptacle on at least one of the upper layer, the base layer, or the mid layer of the tri-layer orthotic.

20. The orthotic system of claim 19, comprising:
a tray for holding the tuning element prior to coupling the tuning element to at least one of the upper layer, the base layer, or the mid layer,
wherein the tray includes a groove to hold a plurality of different configurable tuning elements, wherein an inner profile of the groove matches an inner profile of the receptacle on at least one of the upper layer, the base layer, or the mid layer of the tri-layer orthotic,
wherein the tuning element is labeled with an identifier, and
wherein the tri-layer orthotic comprises a corresponding label at the receptacle configured to receive the tuning element labeled with the identifier.
